(19) United States
(12) Patent Application Publication
(10) Pub. No.: US 2007/0128232 A1
(43) Pub. Date: Jun. 7, 2007

(54) Emulsified perfume oils

(76) Inventor: Wilfried Rahse, Dusseldorf (DE)

Correspondence Address: PAUL & PAUL, 2000 MARKET STREET, PHILADELPHIA, PA, 19103-3229, US

(21) Appl. No.: 11/436,483

(22) Filed: May 18, 2006

Related U.S. Application Data
(63) Continuation of application No. PCT/EP04/10977, filed on Oct. 1, 2004.

(30) Foreign Application Priority Data
Nov. 21, 2003 (DE) 103 54 564.6

(51) Int. Cl. A61K 8/19; A61K 8/31; A61K 8/37; C11D 3/50

(52) U.S. Cl. 424/401; 510/101; 512/1

(57) ABSTRACT

A fragrance and perfume oil concentrate in the form of aqueous emulsion is comprised of at least 30% by weight of perfume oil(s), where the content of the two components water and perfume oil(s) exceeds a value of together 96% by weight. The perfume emulsion is not combustible or flammable and can, therefore, be handled without safety concerns during further processing.

### CROSS-REFERENCE TO RELATED APPLICATIONS

This application is a continuation under 35 U.S.C. § 365(c) and 35 U.S.C. § 120 of International Application PCT/EP2004/010977, filed Oct. 1, 2004. This application also claims priority under 35 U.S.C. § 119 of German Application DE 103 54 564.6, filed Nov. 21, 2003. Each application is incorporated herein by reference in its entirety.

### STATEMENT REGARDING FEDERALLY SPONSORED RESEARCH OR DEVELOPMENT

Not Applicable

### INCORPORATION-BY-0REFERENCE OF MATERIAL SUBMITTED ON A COMPACT DISC

Not Applicable

### BACKGROUND OF THE INVENTION

(1) Field of the Invention

The invention relates to perfume oil concentrates in the form of aqueous emulsions which have a minimum content of perfume of 30% by weight, where the content of the two components water and perfume oil(s) exceeds a value of together 96% by weight. In addition, the invention relates to a method of producing such compositions, and also to products which include such perfume oil concentrates.

Perfume oils are usually only slightly soluble in water. To incorporate them into aqueous preparations, so-called solubilizers or solvents, e.g., lower alcohols, are therefore usually used. There is also the option of emulsifying perfume oils.

Due to their high volatility and their sometimes strong polarity, the emulsification of fragrances is extremely problematic and usually requires the co-use of water-soluble organic solvents, or else the use of very large amounts of emulsifiers.

(2) Description of Related Art, Including Information Disclosed Under 37 C.F.R. §§ 1.97 and 1.98.

German laid-open specification DE 196 24 051 A1 discloses emulsified fragrances in the form of transparent emulsions, the droplet size of which is between 10 and 100 nm. These emulsions are obtainable if the perfume oil is emulsified together with a special co-oil component using alkyl glycosides, the resulting emulsions having a content of up to 50% by weight of a perfume oil, of from 1 to 10% by weight of a co-oil component and from 1 to 30% by weight of an emulsifier of the alkyl glycoside type, with the provision that the resulting emulsions comprise at least 10% of the amount of co-oil component in the perfume. If, for example, 50% by weight of perfume oil are present in the emulsion, then at least 5% by weight of co-oil component must likewise be present. In the case of a content of 40% by weight of perfume oil, accordingly at least 4% by weight of co-oil component are present. In addition, in each case there is also at least 1% by weight of emulsifier.

This means that the perfume oil emulsions according to DE 196 24 051 A1 have a very high content of additives (emulsifiers, coemulsifiers, co-oil component) which adversely effect the natural purity of the perfume oil/water system. In the least favorable case, up to 50% by weight of these additives are present in the perfume oil emulsions described therein. In the most favorable theoretically calculable case, when considering concentrated perfume oil emulsions, i.e. those emulsions which include a perfume oil content of at least 30% by weight, based on the composition, at least 4% by weight of additives are still present in the emulsions, namely at least 3% by weight of co-oil component and at least 1% by weight of emulsifier. However, DE 196 24 051 A1 explicitly discloses only a single concentrated perfume oil emulsion with a perfume oil content of 30% by weight. However, this emulsion includes 11.9% by weight of additives other than water or perfume oil. All of the other emulsions disclosed therein are not concentrated, thus have a perfume oil content of less than 30% by weight and nevertheless include at least 10% by weight of additives other than water or perfume oil. The additives are undesired in many areas since they reduce the natural purity of the system, which, for example, can bring about a change in the scent impression of the perfume oil emulsion or even lead to incompatibility reactions in people who are at high risk for developing allergies.

There was therefore a need for concentrated perfume oil emulsions which are present in relatively pure form.

BRIEF SUMMARY OF THE INVENTION.

One aspect of the present invention pertains to a fragrance and perfume oil concentrate in the form of aqueous emulsions comprising at least 30% by weight of perfume oil(s), where the content of the two components water and perfume oil(s) exceeds a value of together 96% by weight, preferably 97% by weight, advantageously 98% by weight, very advantageously 99% by weight, but in particular, 99.5% by weight, based on the total concentrate. Another aspect of the present invention pertain to a method of producing a concentrate of claim 1 comprising the steps of:
a) adding a thickener to water while stirring the mixture in a first vessel,
b) adding an emulsifier to perfume oil with stirring in a second vessel,
c) adding the contents of the second vessel to the mixture of the first vessel with homogenization.

The resulting emulsion is advantageously not combustible or flammable, can consequently be handled without problems and be further processed. This is very advantageous since the original perfume oils generally have a markedly low flashpoint and therefore present problems during storage, processing and handling. Thus, perfume oils are generally stored only in small amounts and have to be handled by particularly trained personnel. As a result of their low flashpoint, a number of perfume oils cannot be used or handled industrially at all without implementing great complexity. By contrast, handling the composition according to the invention is entirely problem-free and without relatively great odor nuisance.

### BRIEF DESCRIPTION OF THE SEVERAL VIEWS OF THE DRAWING(S)

Not Applicable

### DETAILED DESCRIPTION OF THE INVENTION

In a preferred embodiment, the emulsions have average droplet sizes (d₅₀) which are in a range of from greater than 0.1 μm to equal to or less than 5 μm. It has been found that especially such a droplet size range leads to particularly stable perfume oil concentrates or emulsions. The average droplet size (d₅₀) is the characteristic value at which the cumulative distribution of the droplet diameter assumes the value 0.5=50%. e.g., a d₅₀ of a μm means that, of the material under consideration, 50 (mass)% of the droplets have a diameter greater than a μm and 50 (mass)% have a smaller diameter than a μm.

However, for some applications, particularly in the cosmetics sector, it may also be preferred if the emulsions have smaller average droplet sizes (d₅₀) and are in the form of nanoemulsions. Accordingly, according to another preferred embodiment, the droplet size d₅₀ of the emulsion is not greater than 400 nm, preferably not greater than 300 nm, advantageously not greater than 250 nm, further advantageously not greater than 200 nm, yet more advantageously not greater than 150 nm, in particular, a value of 100 nm is not exceeded. Microemulsions according to the invention with a droplet size d₅₀ of not less than 10 nm, preferably not less than 25 nm, advantageously not less than 40 nm, in particular, not falling below a value of 60 nm, are very particularly preferred here and constitute a particularly advantageous embodiment of the invention.

Nanoemulsions and their production have already been described in the patent literature. An overview of the production and use of nanoemulsions and microemulsions is given by H. Eicke in SÖFW-Journal, 118, 311 (1992) and Th. Förster et al., in SÖFW-Journal, 122, 746 (1996).

According to a preferred embodiment, the compositions comprise at least 40% by weight, preferably at least 50% by weight, advantageously at least 53% by weight, in particular, at least 55% by weight, particularly advantageously at least 60% by weight, of perfume oil(s).

Surprisingly, it has been found that compositions with such contents of perfume oil likewise constitute particularly stable perfume oil concentrates or emulsions.

The perfume oil content should, for reasons of stability, however, preferably also not be too high, meaning that, according to a preferred embodiment, the composition comprises equal to or less than 90% by weight of perfume oil(s).

Perfume oils and fragrances which can be used are individual odorant compounds, e.g., the synthetic products of the ester type, ether type, aldehyde type, ketone type, alcohol type and hydrocarbon type. Odorant compounds of the ester type are, for example, benzyl acetate, phenoxyethyl isobutyrate, p-tert-butyl cyclohexylacetate, linalyl acetate, dimethyl benzylcarbinyl acetate, phenylethyl acetate, linalyl benzoate, benzyl formate, ethyl methylphenylglycinate, allyl cyclohexylpropionate, styrallylpropionat and benzyl salicylate. The ethers include, for example, benzyl ethyl ether, the aldehydes include, for example, the linear alkanals having 8-18 carbon atoms, citral, citronellal, citronellyloxyacetaldehyde, cyclamenaldehyde, hydroxycitronellal, lilial and bourgeonal, the ketones include, for example, the ionones, α-isomethylionone and methyl cedryl ketone, the alcohols include anethol, citronellol, eugenol, geraniol, linalool, phenylethyl alcohol and terpineol, the hydrocarbons include primarily the terpenes and balsams. However, preference is given to using mixtures of different odorants which together produce a pleasing scent note.

The perfume oils or fragrances can also comprise natural odorant mixtures, as are obtainable from vegetable or animal sources, e.g., pine oil, citrus oil, jasmine oil, lily oil, rose oil or ylang ylang oil. Essential oils of lower volatility, which are mostly used as aroma components, are also suitable as perfume oils, e.g., sage oil, chamomile oil, oil of cloves, melissa oil, mint oil, cinnamon leaf oil, linden blossom oil, juniper berry oil, vetiver oil, olibanum oil, galbanum oil and ladanum oil.

The water content of the perfume oil concentrate should, for reasons of stability, likewise preferably not be too high, meaning that, according to a preferred embodiment, the composition comprises less than 60% by weight, advantageously less than 50% by weight, in particular, less than 40% by weight, of water.

The compositions according to the invention also preferably comprise an emulsifier or emulsifiers.

According to a preferred embodiment of the invention, the emulsifier is chosen from the group of nonionic, zwitterionic, ampholytic, cationic and/or anionic emulsifiers.

Suitable emulsifiers are, for example, the emulsifiers listed in the “International Cosmetic Ingredient Dictionary and Handbook”, 7th edition, Volume 2 in the section ‘Surfactants’, in particular, in the subsection ‘Surfactants-Emulsifying Agents’. The term emulsifier here means the totality of the interfacial-active auxiliaries for producing and stabilizing emulsions, meaning that thus within the scope of this invention the term “coemulsifiers” is included in the generic term emulsifier. The emulsifiers sometimes referred to in other specifications as “coemulsifiers” are usually characterized by a predominance of the hydrophobic molecular moiety. They are therefore usually somewhat less soluble in water, can have a tendency to form gels and lamellar liquid crystals and thus increase the viscosity of an emulsion.

Zwitterionic emulsifiers is preferably the term used to refer to those surface-active compounds which carry at least one quaternary ammonium group and at least one carboxylate and one sulfonate group in the molecule. Particularly suitable zwitterionic emulsifiers are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines each having 8 to 18 carbon atoms in the alkyl or acyl group, and cocoacylaminoethylhydroxyethyl carboxymethylglycinate. Particular preference is given to the fatty acid amide derivative known under the CTFA name “Cocamidopropyl Betaine”.

Ampholytic emulsifiers are understood as meaning those surface-active compounds which, apart from a C8/18-alkyl or -acyl group in the molecule, contain at least one free amino group and at least one —COOH or —SO₃H group and are capable of forming internal salts. Examples of suitable ampholytic emulsifiers are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids each having about 8 to 18 carbon atoms in the alkyl group. Particularly preferred ampholytic emulsifiers are N-cocoalkylaminopropionate, cocoacylaminoethylaminopropionate and C12/18-acylsarcosine.

Nonionic emulsifiers are preferably chosen from at least one of the following classes of substances:
alkoxylated fatty acid alkyl esters of the formula R¹CO—(OCH₂CHR²)ₓOR³, in which R¹CO is a linear or branched, saturated and/or unsaturated acyl radical having 6 to 22 carbon atoms, R² is hydrogen or methyl, R³ is linear or branched alkyl radicals having 1 to 4 carbon atoms and x is numbers from 1 to 20,
addition products of ethylene oxide onto fatty acid alkanolamides and fatty amines,
fatty acid N-alkylglucamides,
C₈-C₂₂-alkylamine N-oxides,
alkyl polyglycosides corresponding to the general formula RO-(Z)ₓ where R is a C₈-C₁₆-alkyl group, Z is sugar, and x is the number of sugar units. The alkyl polyglycosides which can be used according to the invention can contain only one specific alkyl radical R. However, these compounds are usually produced starting from natural fats and oils or mineral oils. In this case, mixtures corresponding to the starting compounds or corresponding to the particular work-up of these compounds are present as alkyl radicals R. Particular preference is given to those alkyl polyglycosides in which R consists essentially of C₈- and C₁₀-alkyl groups, essentially of C₁₂- and C₁₄-alkyl groups, essentially of C₈- to C₁₆-alkyl groups or essentially of C₁₂- to C₁₆-alkyl groups.

Any mono- or oligosaccharides can be used as sugar building block Z. Usually, sugars with 5 or 6 carbon atoms, and the corresponding oligosaccharides are used, for example glucose, fructose, galactose, arabinose, ribose, xylose, lyxose, allose, altrose, mannose, gulose, idose, talose, and sucrose. Preferred sugar building blocks are glucose, fructose, galactose, arabinose and sucrose; glucose is particularly preferred. The alkyl polyglycosides which can be used according to the invention contain, on average, 1.1 to 5, preferably 1.1 to 2.0, particularly preferably 1.1 to 1.8, sugar units. The alkoxylated homologs of the specific alkyl polyglycosides can also be used according to the invention. On average, these homologs can contain up to 10 ethylene oxide and/or propylene oxide units per alkyl glycoside unit.

Further nonionic emulsifiers suitable according to the invention are preferably chosen from the addition products of from 4 to 100 ethylene oxide units onto likewise hydrogenated mono-, di- and triglycerides of C₈₋₂₂ fatty acids, the addition products of from 5 to 40 ethylene oxide units onto C₈₋₂₂ fatty alcohols, which is particularly preferred, and the addition products of from 2 to 50 ethylene oxide units and 2 to 35 propylene oxide units onto C₃-C₅-alkanols. Examples of ethoxylated mono-, di- and triglycerides of C₈₋₂₂ fatty acids having 4 to 60 ethylene oxide units are hydrogenated ethoxylated castor oil (INCI name e.g., PEG40 Hydrogenated Castor Oil), olive oil ethoxylate (INCI name: PEG-10 Olive Glycerides), almond oil ethoxylate, mink oil ethoxylate, polyoxyethylene glycol caprylic/capric glycerides, polyoxyethylene glycerol monolaurate and polyoxyethylene glycol coconut fatty acid glycerides. Examples of suitable ethoxylated C₈₋₂₂ fatty alcohols are laureth-12, laureth-23, trideceth-8, ceteareth-12, ceteareth-15, ceteareth-20, ceteareth-30, steareth-10, steareth-15, steareth-20, steareth-30, steareth-40, oleth-10 or oleth-20. Examples of suitable polyethylene glycol-polypropylene glycol mixed ethers of C₃-C₅-alkanols are the PEG-PPG adducts of 1-propanol, 2-propanol and isopropanol, 1-butanol, 2-butanol, isobutanol and 1-pentanol, 2-pentanol and amyl alcohol with 2-50, preferably 4-40, ethylene oxide units and 2-35, preferably 4-30, propylene oxide units, in particular, PPG-28-buteth-35, PPG-26-buteth-26, PPG-5-buteth-5, PPG-25-buteth-25, PPG-5-buteth-20, PPG-33-buteth-45, PPG-20-buteth-30 or PPG-12-buteth-16.

Likewise preferred nonionic emulsifiers are also the ethylene oxide adducts of linear C₃-C₂₂-alcohols with an average number of ethylene oxide units of 1-30. Preferably suitable nonionic emulsifiers are ethylene oxide adducts of branched C₃-C₂₈-alcohols, in particular, of so-called Guerbet alcohols, with an average number of ethylene oxide units of 1-30.

Further preferably suitable nonionic emulsifiers are ethylene oxide-propylene oxide mixed adducts of linear C₃-C₂₂-alcohols with an average number of ethylene oxide units of 2-50, preferably 4-40 and an average number of propylene oxide units of 2-35, preferably 4-30.

Particularly preferred nonionic emulsifiers are propylene oxide adducts of linear C₃-C₂₂-alcohols. The average number of propylene oxide units is 1-30, preferably 5-25 and particularly preferably 8-15. Suitable propoxylated emulsifiers are, for example, PPG-3 myristyl ether (Witconol®APM), PPG-14 butyl ether (Ucon Fluid®AP), PPG-15 stearyl ether (Arlamol®E), PPG-9 butyl ether (Breox® B25) and PPG-10-butanediol (Macol® 57), where PPG-14 butyl ether and PPG-15 stearyl ether are particularly preferred.

Preferably, the emulsified perfume oils can comprise at least one nonionic emulsifier with an HLB value of from 3 to 18, according to the definitions listed in the Römpp-Lexikon Chemie (ed. J. Falbe, M. Regitz), 10th edition, Georg Thieme Verlag Stuttgart, New York (1997), page 1764. Nonionic O/W emulsifiers with an HLB value of 10-15 and nonionic W/O emulsifiers with an HLB value of 3-6 may be particularly preferred according to the invention.

In a particularly preferred embodiment, exclusively nonionic emulsifiers are present, preferably only a single nonionic emulsifier, advantageously chosen from the addition products of from 5 to 40 ethylene oxide units onto C₈₋₂₂ fatty alcohols, in particular, Eumulgin® B3 (cetylstearyl alcohol+30-EO; obtainable via Cognis Deutschland GmbH). Within the scope of this particularly preferred embodiment, likewise most preferred nonionic emulsifiers are the ethoxylated fatty acid alkanolamides, preferably ethoxylated coconut fatty acid monoethanolamides, in particular, coconut fatty acid monoethanolamides plus 4 ethylene oxide units, which corresponds, for example, to the commercial product Eumulgin® C4 (obtainable via Cognis Deutschland GmbH). When using emulsifiers corresponding to these preferred embodiments, in particular, when using Eumulgin® B3 and/or Eumulgin® C4, it is possible to produce perfume oil concentrates according to the invention with excellent stability.

Nonfoaming emulsifiers are exceptionally preferred.

Likewise suitable according to the invention are cationic emulsifiers, preferably of the quaternary ammonium compound type, the esterquat type and the amidoamine type.

Preferred quaternary ammonium compounds are ammonium halides, in particular, chlorides and bromides, such as alkyltrimethylammonium chlorides, dialkyldimethylammonium chlorides and trialkylmethylammonium chlorides, e.g., cetyltrimethylammonium chloride, stearyltrimethylammonium chloride, distearyldimethylammonium chloride, lauryidimethylammonium chloride, lauryldimethylbenzylammonium chloride and tricetylmethylammonium chloride, and the imidazolium compounds known under the INCI names Quaternium-27 and Quaternium-83. The long alkyl chains of the above-mentioned emulsifiers preferably have 10 to 18 carbon atoms.

Esterquats are known substances which contain both at least one ester function and also at least one quaternary ammonium group as structural element. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are sold, for example, under the trade name Stepantex®, Dehyquart® and Armocare®. The products Armocare® VGH-70, an N,N-bis(2-palmitoyloxyethyl)dimethylammonium chloride, and Dehyquart® F-75, Dehyquart® C4046, Dehyquart® L80 and Dehyquart® AU-35 are examples of such esterquats.

The alkylamidoamines are usually produced by amidation of natural or synthetic fatty acids and fatty acid cuts with dialkylaminoamines. One compound from this group of substances which is particularly suitable according to the invention is the stearamidopropyldimethylamine available commercially under the name Tegoamid® S 18.

The major advantage of the cationic emulsifiers is that they impart a positive charge to the emulsion droplets and in so doing bring about increased adsorption of such perfume oils from the emulsion phase onto negatively charged surfaces, e.g., on textile fibers.

Anionic emulsifiers preferably have a water-solubilizing, anionic group, such as, for example, a carboxylate, sulfate, sulfonate or phosphate group and a lipophilic alkyl group having about 8 to 30 carbon atoms. In addition, glycol or polyglycol ether groups, ester, ether and amide groups and also hydroxyl groups may be present in the molecule. Examples of suitable anionic emulsifiers are, in each case in the form of the sodium, potassium and ammonium and also the mono-, di- and trialkanolammonium salts having 2 to 4 carbon atoms in the alkanol group,
linear and branched fatty acids having 8 to 30 carbon atoms (soaps),
ether carboxylic acids of the formula R—O—(CH₂—CH₂O)ₓ—CH₂—COOH, in which R is a linear alkyl group having 8 to 30 carbon atoms and x=0 or 1 to 16,
acyl sarcosides having 8 to 24 carbon atoms in the acyl group,
acyl taurides having 8 to 24 carbon atoms in the acyl group,
acyl isethionates having 8 to 24 carbon atoms in the acyl group,
acyl glutamates of the formula (I), 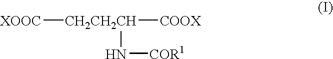
in which R¹CO is a linear or branched acyl radical having 6 to 22 carbon atoms and 0, 1, 2 or 3 double bonds and X is hydrogen, an alkali metal and/or alkaline earth metal, ammonium, alkylammonium, alkanolammonium or glucammonium, for example acyl glutamates which are derived from fatty acids having 6 to 22, preferably 12 to 18, carbon atoms, such as, for example, C_{12/14} or C_{12/18} coconut fatty acid, lauric acid, myristic acid, palmitic acid and/or stearic acid, in particular, sodium N-cocoyl- and sodium N-stearoyl-L-glutamate,
esters of a hydroxy-substituted di- or tricarboxylic acid of the general formula (II), 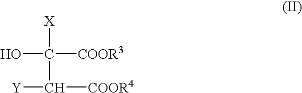
in which X═H or a —CH₂COOR² group, Y═H or —OH, with the proviso that Y═H if X═-CH₂COOR², R², R³ and R⁴, independently of one another, are a hydrogen atom, an alkali metal or alkaline earth metal cation, an ammonium group, the cation of an ammonium-organic base or a radical Z which originates from a polyhydroxylated organic compound which are chosen from the group of etherified (C₆-C₁₈)-alkyl polysaccharides having 1 to 6 monomeric saccharide units and/or etherified aliphatic (C₆-C₁₆)-hydroxyalkylpolyols having 2 to 16 hydroxyl radicals, with the proviso that at least one of the groups R², R³ or R⁴ is a radical Z,
esters of sulfosuccinic acid or of sulfosuccinates of the general formula (III), 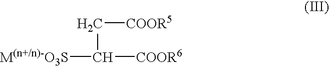
   in which M^{(n+/n)} when n=1 is a hydrogen atom, an alkali metal cation, an ammonium group or the cation of an ammonium-organic base and when n=2, an alkaline earth metal cation, and R¹ and R⁶, independently of one another, are a hydrogen atom, an alkali metal or alkaline earth metal cation, an ammonium group, the cation of an ammonium-organic base or a radical Z which originates from a polyhydroxylated organic compound which is chosen from the group of etherified (C₆-C₁₈)-alkyl polysaccharides having 1 to 6 monomeric saccharide units and/or etherified aliphatic (C₆-C₁₆)-hydroxyalkyl polyols having 2 to 16 hydroxyl radicals, with the proviso that at least one of the groups R⁵ or R⁶ is a radical Z,
sulfosuccinic mono- and dialkyl esters having 8 to 24 carbon atoms in the alkyl group and sulfosuccinic monoalkylpolyoxyethyl esters having 8 to 24 carbon atoms in the alkyl group and 1 to 6 oxyethyl groups,
linear alkanesulfonates having 8 to 24 carbon atoms,
linear alpha-olefinsulfonates having 8 to 24 carbon atoms,
alpha-sulfo fatty acid methyl esters of fatty acids having 8 to 30 carbon atoms,
alkyl sulfates and alkyl polyglycol ether sulfates of the formula R—(O—CH₂—CH₂)ₓ—OSO₃H, in which R is a preferably linear alkyl group having 8 to 30 carbon atoms and x=0 or 1-12,
mixed surface-active hydroxysulfonates as in DE-A-37 25 030,
esters of tartaric acid and citric acid with alcohols, which constitute addition products of about 2-15 molecules of ethylene oxide and/or propylene oxide onto C₈₋₂₂ fatty alcohols,
alkyl and/or alkenyl ether phosphates,
sulfated fatty acid alkylene glycol esters,
monoglyceride sulfates and monoglyceride ether sulfates.

Preferred anionic emulsifiers are acyl glutamates, acyl isethionates, acyl sarcosinates and acyl taurates, each with a linear or branched acyl radical having 6 to 22 carbon atoms and 0, 1, 2 or 3 double bonds, which, in particularly preferred embodiments, is chosen from an octanoyl, decanoyl, lauroyl, myristoyl, palmitoyl and stearoyl radical, esters of tartaric acid, citric acid or succinic acid or of salts of these acids with alkylated glucose, in particular, the products with the INCI name Disodium Coco-Glucoside Citrate, sodium coco-glucoside tartrate and disodium coco-glucoside sulfosuccinate, alkyl polyglycol ether sulfates and ether carboxylic acids having 8 to 18 carbon atoms in the alkyl group and up to 12 ethoxy groups in the molecule, sulfosuccinic mono- and dialkyl esters having 8 to 18 carbon atoms in the alkyl group and sulfosuccinic monoalkylpolyoxyethyl esters having 8 to 18 carbon atoms in the alkyl group and 1 to 6 ethoxy groups.

The content of emulsifiers in the overall composition is very small due to the high fraction of the components water and perfume oil(s); according to a preferred embodiment, however, it is in the range of from at least 0.1% by weight to equal to or less than 4% by weight; preferably less than 2.5% by weight, advantageously less than 2.0% by weight, very advantageously less than 1.5% by weight, exceptionally advantageously less than 1.0% by weight, but in particular, less than 0.7% by weight, based on the total composition.

According to a preferred embodiment, a minimum amount of emulsifier of 0.12% by weight, preferably of 0.175% by weight, advantageously of 0.2% by weight, based on the total composition.

It is particularly advantageous to make do with such small amounts of emulsifier since the emulsified perfume oil can in this way also be further used without problems for applications in which emulsifiers are in some circumstances only desired in very small amounts, for example in the case of some cosmetic uses, e.g., those dealing with irritated skin.

The above statements with regard to the emulsifiers also apply to the nanoemulsions according to the invention. However, there are here further advantageous embodiments which lead to a further increase in the stability of the emulsion. It is advantageous here if a nanoemulsion according to the invention has at least two emulsifiers. According to a preferred embodiment, a nanoemulsion according to the invention comprises an emulsifier system from at least one lipophilic, preferably lipophilic cationic, emulsifier, and at least one hydrophilic, preferably hydrophilic nonionic, emulsifier.

If, in the case of the nanoemulsions according to the invention, a cationic and a nonionic emulsifier is present at the same time, then the quantitative ratio of cationic emulsifier to nonionic emulsifier is advantageously in the range from 70:1 to 1:3, in particular, from 30:1 to 1:2, preferably from 10:1 to 1:1, and particularly preferably from 5:1 to 2:1.

For the purposes of this application, emulsifiers are classed as lipophilic essentially when they have firstly HLB values of less than or equal to 8 and when they, secondly, are advantageously predominantly soluble in C₁₂-C₂₀ triglycerides, or are miscible therewith. Lipophilicity can arise, inter alia, for example when the emulsifiers, for example, have hydrocarbon radicals having 6 to 22 carbon atoms or, for example, contain aryl radicals, to give illustrative, but nonlimiting examples. Lipophilic emulsifiers have essentially a less polar, more apolar character. Preferred lipophilic emulsifiers for the purposes of the invention constitute lipophilic, ethoxylated fatty alcohols (C₁₂-C₂₀-fatty alcohols having 1 to 3 EO units). Ethylene oxide/propylene oxide-modified silicone oil emulsifiers are also suitable.

Accordingly, for the purposes of this application, emulsifiers are then essentially regarded as being hydrophilic if they firstly have an HLB value of greater than/equal to 13 and if they, secondly, are advantageously predominantly soluble in water or miscible therewith. Hydrophilic emulsifiers essentially have a polar character. Hydrophilicity can arise, inter alia, for example when the emulsifier contains, for example, hydroxy group(s), ester group(s), ether group(s) or glycerol group(s), to give illustrative, but nonlimiting examples.

The term HLB value is known to the person skilled in the art. The HLB value is a measure introduced by Griffin (1950) of the water and oil solubility of surfactants or emulsifiers and the stability of emulsions. Experimentally, the HLB value can be determined, for example, by the phenol titration method by adding 5% strength phenol solution to the surfactant or emulsifier solution until cloudy. In addition, the HLB value can be determined by means of (gas) chromatography, by determining the dielectric constant or by means of colorimetry. Detailed information on this and also lists of the HLB values of commercial emulsifiers are given in the relevant specialist literature or in reference works such as, for example, Kirk-Othmer Encyclopedia of Chemical Technology by John Wiley & Sons. The HLB scale usually ranges from 1 to 20. Substances with a low HLB value (3 to 8) are generally regarded as being lipophilic and generally considered to be good W/O emulsifiers, whereas substances with a higher HLB value (8 to 18) are considered to be hydrophilic and generally act as O/W emulsifiers.

It is particularly advantageous if the nonionic hydrophilic emulsifier present in the nanoemulsion is chosen from ethoxylated fatty alcohols and/or ethoxylated fatty acid alkanolamides. With regard to the ethoxylated fatty alcohols, particular preference here is given to the addition products of from 5 to 40 ethylene oxide units onto C₈₋₂₂-fatty alcohols, with Eumulgin® B3 (cetylstearyl alcohol+30 EO; obtainable via Cognis Deutschland GmbH) in particular, being extremely preferred. With regard to the ethoxylated fatty acid alkanolamides, particular preference is preferably given to the ethoxylated coconut fatty acid monoethanolamides, in particular, coconut fatty acid monoethanolamides plus 4 ethylene oxide units, which corresponds, for example, to the commercial product Eumulgin® C4 (obtainable via Cognis Deutschland GmbH).

It is likewise particularly advantageous if the cationic emulsifiers present in the nanoemulsion are quaternary ammonium compounds, advantageously alkylated quaternary ammonium compounds, preferably with one, two or three hydrophobic groups which are joined to a quaternized di- or triethanolamine or an analogous compound in particular, via ester or amido bonds. For example, N-methyl-N(2-hydroxyethyl)-N,N-(ditallow-acyloxyethyl)ammonium methosulfate or N-methyl-N(2-hydroxyethyl)-N,N-(dipalmitoylethyl)ammonium methosulfate are very advantageous.

For the nanoemulsion too, it is the case according to the invention that the content of emulsifiers in the overall composition is very small. According to a preferred embodiment, a nanoemulsion according to the invention comprises not more than 3.5% by weight, preferably not more than 3% by weight, advantageously not more than 2.5% by weight, in particular, not more than 2% by weight, but at least 0.1% by weight, of lipophilic emulsifiers. A nanoemulsion according to the invention likewise comprises, according to a preferred embodiment, not more than 3.5% by weight, preferably not more than 3% by weight, advantageously not more than 2.5% by weight, in particular, not more than 2% by weight, but at least 0.1% by weight of hydrophilic emulsifiers.

Preferably, the composition according to the invention also comprises thickeners. The content of thickeners in the overall composition is likewise very small due to the large content of the components water and perfume oil(s), according to a preferred embodiment, however, it is in the range of from at least 0.1% by weight, but less than 4% by weight, preferably less than 2.5% by weight, advantageously less than 1.9% by weight, very advantageously less than 1.5% by weight, extremely advantageously less than 1.0% by weight, but in particular, less than 0.7% by weight, based on the total composition.

According to a preferred embodiment, suitable thickeners are chosen from the group of
a) polysaccharides, in particular, xanthan gum, guar derivatives, gum arabic, karaya gum, tragacanth, tara gum, gellan, carrageen, carob seed flour, agar agar, alginates, pectins and/or dextrans,
b) organic completely synthetic thickeners, in particular, polyacrylates, polyacrylamides, polyvinylpyrrolidone, polvinyl alcohol, polyethylene glycols, hydrophobically modified polyethers, polyurethanes, styrene-maleic anhydride copolymers, salts thereof and/or derivatives thereof,
c) cellulose derivatives, in particular, hydroxyethylcellulose, carboxymethylcellulose, hydroxypropylmethylcellulose, hydroxypropylcellulose; ethylhydroxyethylcellulose, methylcellulose,
d) starch fractions and derivatives, in particular, amylose, amylopectin and dextrins,
e) clays, in particular, bentonite.

In the case of the nanoemulsions according to the invention, it is also advantageous if at least 0.05% by weight, preferably at least 0.1% by weight, advantageously at least 0.15% by weight, in particular, at least 0.2% by weight, but not more than 3% by weight, preferably not more than 2.5% by weight, advantageously not more than 2.0% by weight, very advantageously not more than 1.5% by weight, yet more advantageously not more than 1.0% by weight, extremely advantageously not more than 0.75% by weight, most advantageously not more than 0.5% by weight of thickeners is present.

In contrast to the normal emulsions, on account of the droplet fineness, the nanoemulsions are advantageously so stable that a thickener is preferably not necessary. In a preferred embodiment concerning emulsions with a droplet diameter d₅₀ of <200 nm, the addition of thickeners is dispensed with.

According to a preferred embodiment, for the normal emulsion, a minimum amount of thickener in the composition of 0.12% by weight, preferably of 0.2% by weight, based on the weight of the composition.

For the purposes of the invention, the co-effect of lower alcohols can preferably be very largely dispensed with. The concentrates according to the invention are therefore preferably essentially free from lower alcohols. If relatively small amounts, e.g., through the perfume oils themselves or through other raw materials, e.g., through the cationic emulsifiers, pass into the concentrates, the content of such alcohols having 1-4 carbon atoms in the concentrate should preferably be less than 1% by weight.

The compositions according to the invention have the advantage that they can comprise perfume oils in very high concentration, preferably in concentrations up to 90% by weight of perfume oil, based on the overall composition. One benefit of the high perfume oil concentration is, for example, that the emulsified perfume oils require only negligibly more storage space coupled with a significantly reduced expenditure on safety compared with the original perfume oils which are very disadvantageous in terms of handling.

In the Applicant's view, the best configuration of the invention for normal emulsions at the time of the application consists in those compositions according to the invention which, besides the two fundamental components perfume oil and water, comprise at least one nonionic emulsifier, preferably only a single nonionic emulsifier, which is advantageously chosen from the addition products of from 5 to 40 ethylene oxide units onto C₈₋₂₂ fatty alcohols, in particular, Eumulgin® B3 (cetylstearyl alcohol+30 EO; obtainable via Cognis Deutschland GmbH) and/or the ethoxylated fatty acid alkanolamides, preferably ethoxylated coconut fatty acid monoethanolamides, in particular, coconut fatty acid monoethanolamide plus 4 ethylene oxide units, which corresponds, for example, to the commercial product Eumulgin® C4 (obtainable via Cognis Deutschland GmbH). In this best configuration, at least one thickener is likewise present, preferably only a single thickener, advantageously chosen from the group of polysaccharides, including, in particular, those chosen from xanthan gum, guar derivatives, gum arabic, karaya gum, tragacanth, tara gum, gellan, carrageen, carob seed flour, agar agar, alginates, pectins and/or dextrans, but xanthan gum is most advantageous, and/or chosen from the group of cellulose derivatives, including preferably those chosen from hydroxyethylcellulose, carboxymethylcellulose, hydroxypropylmethylcellulose, hydroxypropylcellulose, ethylhydroxyethylcellulose, methylcellulose, but hydroxyethylcellulose is the most advantageous. In this best configuration, the perfume oil concentrate comprises no other emulsifiers and thickeners than those mentioned above. In this best configuration, the perfume oil concentrate advantageously comprises less than 2.0% by weight, but preferably at least 0.1% by weight, in each case of thickener and also of emulsifier. In this best configuration, the perfume oil concentrate comprises preferably at least 40% by weight of perfume oil and advantageously less than 60% by weight, in particular, less than 50% by weight, of water.

Last but not least, perfume oil concentrates which satisfy the criteria presented above of the best configuration are characterized by excellent stability.

The invention further provides a product which comprises a perfume oil concentrate according to the invention and at least one active ingredient, auxiliary and/or additive, in particular, chosen from the following list:
hair-conditioning compounds, such as phospholipids, for example soya lecithin, egg lecithin and cephalins, and silicone oils,
dimethyl isosorbide and cyclodextrins,
solvents and solubility promoters, such as ethanol, isopropanol, ethylene glycol, propylene glycol, glycerol and diethylene glycol,
active ingredients which improve fiber structure, in particular, mono-, di- and oligosaccharides, such as, for example, glucose, galactose, fructose, fruit sugar and lactose,
conditioning active ingredients, such as paraffin oils, vegetable oils, e.g., sunflower oil, orange oil, almond oil, wheat germ oil and peach kernel oil, and
quaternized amines, such as methyl-1-alkylamidoethyl-2-alkylimidazolinium methosulfate, antifoams, such as silicones,
dyes for coloring the composition,
antidandruff active ingredients, such as piroctone olamine, zinc omadine and climbazole,
active ingredients, such as allantoin and bisabolol,
cholesterol,
consistency regulators, such as sugar esters, polyol esters or polyol alkyl ethers,
fats and waxes, such as spermeciti, beeswax, montan wax and paraffins,
fatty acid alkanolamides,
complexing agents, such as EDTA, NTA, β-alaninediacetic acid and phosphonic acids,
swelling and penetration substances, such as primary, secondary and tertiary phosphates,
opacifiers, such as latex, styrene/PVP and styrene/acrylamide copolymers
pearlizing agents, such as ethylene glycol mono- and distearate, and PEG-3 distearate,
pigments,
propellants, such as propane/butane mixtures, N₂O, dimethyl ether, CO₂ and air,
antioxidants,
odor enhancers, and
transfer substances.

In a preferred embodiment, the active ingredient(s), auxiliary(ies) and/or additive(s) present is or are present here in such low concentrations that even in the resulting product the content of the two components water and perfume oil(s) exceeds a value of together 96% by weight, based on the total product.

Equally, depending on the desired field of use of the product, it may also be desired for the active ingredient(s), auxiliary(ies) and/or additive(s) to be present in larger amounts in the resulting product, meaning that such products likewise constitute a preferred embodiment with regard to these products.

With regard to other and/or further optional components, reference may be made to the fact that the selection of such components with regard to the provision of corresponding products essentially depends on which intended use the resulting product is to have. If the emulsified perfume oil, for example within the scope of the cosmetic field of use, is to be further processed or be used there, then, as far as the nature and also the amounts of the components to be usefully used therein is concerned, reference is made expressly to the relevant handbooks which are well-known to the person skilled in the art, e.g., the monograph by K. H. Schrader, Grundlagen und Rezepturen der Kosmetika [Fundamentals and formulations of cosmetics], 2nd edition, Hüthig Buch Verlag Heidelberg, 1989. In analogy to this, other relevant handbooks relating to other use purposes and fields of use are well-known to the respective person skilled in the art.

It is preferred if essentially water-soluble active ingredients, auxiliaries and additives are incorporated, for example stirred, into the finished emulsified perfume oil concentrate to give corresponding products.

The invention further provides a method of producing a composition which has the features described above. This method, which is a two-pot method, comprises the steps:
a) addition of thickener to water while stirring the mixture in a first vessel,
b) addition of emulsifier to perfume oil with stirring in a second vessel,
c) addition of the contents of the second vessel to the mixture of the first vessel with homogenization.

According to a preferred embodiment of the method, the addition of the emulsifier to the perfume oil takes place at temperatures below 60° C., advantageously in the temperature range from 25 to 55° C.

According to a further preferred embodiment of the method, the mixture of the first vessel is heated before adding the perfume oil/emulsifier to a temperature below 50° C., preferably below 40° C., advantageously to a temperature in the range from 20-35° C.

The invention further provides a method which is carried out in a single vessel, where firstly the water is introduced, then the thickener and the emulsifier are added, advantageously at the same time, with stirring, and then the perfume oil is added with homogenization such as by means of a homogenization mixer. The advantage of this method is the more advantageous process economics since it is a one-pot method. According to a preferred embodiment, this method is characterized in that the addition of the thickener takes place at temperatures below 60° C., preferably below 50° C., advantageously at temperatures in the range from 15-30° C. and that the addition of the emulsifier takes place during or after the heating of the mixture to a temperature below 70° C., preferably below 60° C., advantageously at temperatures in the range 35-55° C., and that the mixture is cooled before adding the perfume oil to a temperature below 50° C., preferably below 40° C., advantageously to a temperature in the range from 20-35° C.

Despite the advantages of this method, the best method configuration consists in the two-pot method.

According to a further preferred embodiment, the method according to the invention takes place with the addition of gas, the gas preferably being chosen from the group of inert gases, nitrogen and/or carbon dioxide. The addition of gas preferably takes place according to the mixing principle, i.e. by introducing the gas into the liquid mixture. The mixing principle is advantageously to be combined with the pressure-change principle, i.e. a preferably repeated evacuation and aeration of the mixture with the gas is carried out.

By adding the gas, undesired oxidation reactions are avoided and the storage stability of the composition is improved.

The gas is advantageously added to the perfume oil concentrate after the perfume oil concentrate has been prepared. In the case of particularly sensitive formulations or perfume oils, it is likewise advantageous to ensure an addition of gas throughout the entire period of preparation. In the case of the method with 2 vessels, both vessels and the mixtures present therein are advantageously supplied with gas.

According to a further preferred embodiment, following the method for producing the perfume oil concentrate, solids, preferably fine powders, can be emulsified into the finished concentrate, resulting in corresponding products. In particular, these solids are additives customary in detergents which are advantageously chosen from the group of zeolites, bentonites, silicates, phosphates, urea and/or derivatives thereof, sulfates, carbonates, citrate, citric acid, acetate and/or salts of anionic surfactants. Fine powders means that the solids preferably have a d₅₀ value of less than 0.2 mm, advantageously less than 0.1 mm, in particular, less than 0.05 mm.

Accordingly, the invention further provides a product comprising a perfume oil concentrate according to the invention and solids, and preferably solids customary in detergents, advantageously chosen from the group of zeolites, bentonites, silicates, phosphates, urea and/or derivatives thereof, sulfates, carbonates, citrates, citric acid, acetates and/or salts of the anionic surfactants, in particular, in the form of fine powders. Fine powders means that the solids preferably have a d₅₀ value of less than 0.2 mm, advantageously less than 0.1 mm, in particular, less than 0.05 mm.

This invention further provides a product which comprises a perfume oil concentrate according to the invention and a lipophilic thickener, preferably chosen from the group of fatty alcohols, fatty alcohol ethoxylates and/or derivatives thereof, fatty acids, fatty acid alkanolamide ethoxylates, paraffins and/or silicone oils, here the lipophilic thickener is present advantageously in amounts of from 0.05 to 3% by weight, in particular, from 0.1 to 1% by weight, in each case based on the perfume oil concentrate, where such a product is very advantageously characterized in that it has a delayed scent effect. Such thickened compositions are characterized in that, as a consequence of the increased viscosity of the perfume oils, they have a significantly longer scent effect or scent duration than perfume oils which have not been thickened. The scent effect develops here continuously and extends over a significantly prolonged period. For the purposes of this application, thickeners are essentially cast as being lipophilic if they are advantageously predominantly soluble in C₁₂-C₂₀ triglycerides, or are miscible with these. Lipophilicity can arise, inter alia, for example when the thickeners have, for example, hydrocarbon radicals with 6 to 22 carbon atoms or, for example, contain aryl radicals, to give illustrative, but nonlimiting examples. Preferred lipophilic thickeners for the purposes of this invention are the silicone oils. By contrast, for the purposes of this application, thickeners are essentially considered to be hydrophilic when they are advantageously predominantly soluble in water, or are miscible with this. Hydrophilicity can arise inter alia, for example, when the thickener comprises, for example, hydroxy group(s), ester group(s), ether group(s) or glycerol group(s), to give illustrative, but nonlimiting examples.

Accordingly, this invention further provides a method of producing an above-described product where a lipophilic thickener is added to the perfume oil concentrate according to the invention with homogenization before or after emulsification, preferably in amounts of from 0.05 to 3% by weight, in particular, in amounts of from 0.1 to 1% by weight, based on the total composition.

According to a preferred embodiment of this method, in a first vessel, the lipophilic thickener is added to the perfume oil, preferably with stirring and then, at a slightly elevated temperature, the emulsifier is added and dissolved, preferably 0.1 to 1 part by weight of emulsifier based on the perfume oil concentrate consisting of perfume oil emulsion and lipophilic thickener such as, for example, octanol, decanol, dodecanol or silicone oils. This solution, after cooling, is stirred into a second vessel which comprises a mixture of water and hydrophilic thickener, such as, for example, hydroxyethylcellulose, and then homogenized. The advantage of adding the lipophilic thickener before the emulsification is that a very uniform composition of the individual droplets is obtained.

Accordingly, this invention further provides the use of a lipophilic thickener for producing a perfume oil emulsion with delayed scent effect.

The perfume oil concentrates according to the invention can either be diluted with water or added to aqueous preparations without resulting in coalescence of the emulsified perfume oils. This is an important advantage of the compositions according to the invention and opens up far-reaching application perspectives and possible uses.

A particularly important aspect of the invention in terms of application is therefore the use of the perfume oil concentrates according to the invention for the perfuming of aqueous preparations, for example in the form of aqueous solutions or aqueous dispersions, of every type. Such preparations may, for example, be cosmetic compositions, preferably cosmetic cleansing compositions, such as foam bath and shower bath formulations, liquid soaps, shampoos or other aqueous body-cleansing compositions.

The invention therefore further provides a cosmetic product comprising a perfume oil concentrate according to the invention and at least one cosmetically effective substance, preferably chosen from the group of skincare active ingredients.

In a preferred embodiment of the invention, such a product comprises
a) 0.01 to 75% by weight of a composition according to the invention, and
b) at least 0.01% by weight of at least one cosmetically effective substance, preferably chosen from the group of skincare active ingredients,
   where the percentage by weight given is in each case based on the overall product.

Here, the term skincare active ingredients is understood as meaning all those active ingredients which impart a sensory and/or cosmetic advantage to the skin. The skincare active ingredients are preferably chosen from the following substances:
a) Waxes, such as, for example, carnauba, spermaceti, beeswax, lanolin and/or derivatives thereof and others.
b) Hydrophobic plant extracts
c) Hydrocarbons, such as, for example, squalenes and/or squalanes
d) Higher fatty acids, preferably those with at least 12 carbon atoms, for example lauric acid, stearic acid, behenic acid, myristic acid, palmitic acid, oleic acid, linoleic acid, linolenic acid, isostearic acid and/or polyunsaturated fatty acids and others.
e) Higher fatty alcohols, preferably those with at least 12 carbon atoms, for example lauryl alcohol, cetyl alcohol, stearyl alcohol, oleyl alcohol, behenyl alcohol, cholesterol and/or 2-hexadecanol and others.
f) Esters, preferably those such as cetyl octanoate, lauryl lactate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, gylycerol tristearate, alkyl lactate, alkyl citrate and/or alkyl tartrate and others.
g) Lipids, such as, for example, cholesterol, ceramides and/or sucrose esters and others.
h) Vitamins, such as, for example, the vitamins A and E, vitamin alkyl esters, including vitamin C alkyl esters and others.
i) Sunscreens.
j) Phospholipids.
k) Derivatives of alpha-hydroxy acids.
l) Germicides for cosmetic use, either synthetic ones, such as, for example, salicylic acid and/or others, or natural ones, such as, for example, neem oil and/or others.
m) Silicones.
n) Mixtures of any of the above-mentioned components.

A further field of use, in particular, for compositions according to the invention with cationic emulsifiers, is the perfuming of, advantageously aqueous, textile treatment compositions, preferably textile aftertreatment compositions, e.g., of fabric softeners. However, the perfuming of textile treatment liquors themselves can also take place with the help of the perfume oil concentrates according to the invention. The emulsified perfume oils can also be emulsified into liquid detergents. Finally, the perfume oil concentrates can be used for all tasks of perfuming technical and cosmetic products, particularly where no alcoholic preparations can be used.

The invention therefore further provides a product for textile treatment, comprising a composition according to the invention and a substance suitable for the textile treatment.

In a preferred embodiment of the invention, such a product comprises
a) 0.01 to 50% by weight of a composition according to the invention, and
b) 0.1 to 50% by weight of a substance suitable for the textile treatment, preferably washing surfactant, advantageously chosen from the group nonionic, anionic, cationic, zwitterionic and/or ampholytic washing surfactant, and,
c) optionally and preferably up to 50% by weight of one or more washing additives, advantageously chosen from builders, enzymes, brighteners, soil repellents, foam regulators, antistatic agents and/or dispersants,
   where the percent by weight data is in each case based on the overall product.

The compositions according to the invention can also be sprayed without problems. Advantageously, they can also be sprayed onto solid, for example pulverulent or granular, detergents. It is particularly advantageous here that the droplet size of the sprayed-on perfume oil is smaller by about a factor of ten than that of the original perfume oils. The compositions are thus absorbed particularly readily by the solid detergent.

### EXAMPLES

Preparation of Emulsified Perfume Oil

### Example 1

Preparation of 100 g of Emulsion with a Perfume Oil Content of 64.4% by Weight

0.35 g of hydroxyethylcellulose (Natrosol®: source: Hercules Aqualon) were dissolved in 35 g of water at 25° C. with stirring in beaker 1. In beaker 2, 0.25 g of Eumulgin® B3 (cetylstearyl alcohol+30 EO; source: Cognis Deutschland GmbH) was added to 64.4 g of commercially available perfume oil with stirring and heating to about 45° C. until a largely clear solution was obtained. The mixture in beaker 2 was left to cool to 30° C. and then this solution was added to the aqueous phase (beaker 1) over the course of 20 seconds using a laboratory homogenization rod of the Ultraturrax type (Janke und Kunkel). The entire mixture was then homogenized at a maximum speed of 30 s (the average droplet size of the emulsion d₅₀ is about 2 micrometers).

The two components water and perfume oil constitute 99.4% by weight of the perfume oil concentrate in this example.

### Example 2

Preparation of 100 g of Emulsion with a Perfume Oil Content of 50% by Weight

In beaker 1, 0.2 g of xanthan was dissolved in 49.65 g of water, while in beaker 2 0.15 g of Eumulgin® B3 (source: Cognis Deutschland GmbH) was dissolved in 50 g of perfume oil at 45° C. After cooling to 30° C., the contents of beaker 2 were stirred into beaker 1. Homogenization was then carried out using an Ultraturrax at maximum speed for 45 s.

### Example 3

Preparation of 100 g of Emulsion with a Perfume Oil Content of 50%

In beaker 1, 0.2 g of xanthan was dissolved in 49.65 g of water, and in beaker 2 0.15 g of Eumulgin® C4 (coconut fatty acid monoethanolamide +4 EO; source Cognis Deutschland GmbH) was dissolved in 50 g of perfume oil at 45° C. After cooling to 35° C., the contents of beaker 2 were stirred into beaker 1 and then emulsified by means of ultrasound (ultrasound rod from Bandelin, Sonopuls type HD 2200 with SH 225 G). According to measurement by means of laser diffraction (Malvern), the emulsion had an average droplet size d₅₀ of 600 nm.

The two components water and perfume oil constitute 99.65% by weight of the perfume oil concentrate in Examples 2 and 3.

### Example 4

Preparation of 1000 g of Nanoemulsion with a Perfume Oil Content of 50% by Weight.

In a large beaker 1, 463 g of water were initially introduced and 1 g of xanthan was dissolved therein. In beaker 2, 500 g of perfume oil were heated to 45° C. 20 g of Dehydol® LS 2 (C₁₂-C₁₄-fatty alcohol +2 EO, Cognis) and 16 g of Eumulgin® B3 have been suspended and dissolved therein. The contents of beaker 2 were stirred into the water in beaker 1 and then homogenized using the Ultraturrax. After cooling to 30° C., this solution served as starting material for the high-pressure homogenizer (pilot plant machine from Nivo/Soavi), which was passed three times at a pressure of 700 bar at a maximum of 45° C. (regulated via cooling). The nanoemulsion forms in the first pass. The second and third passes were used to minimize the droplet fraction above 200 nm. According to measurement by means of laser diffraction (Malvern), the emulsion had an average droplet size d₅₀ of 110 nm. The smallest droplets were 50 nm, the largest 200 nm. The stability of the emulsion was tested by various methods. Following a storage test at a constant 50° C. over a period of seven days, the microemulsion was just as stable. Following a three-minute centrifuge test at 25° C. and a centrifugal acceleration of 36,000 g, the microemulsion was just as stable.

The two components water and perfume oil constitute 96.3% by weight of the perfume oil concentrate in this example.

1. A fragrance or perfume oil concentrate in the form of an aqueous emulsion, comprising at least 30% by weight of perfume oil(s), wherein the total of the water and perfume oil(s) is greater than 96% by weight, based on the weight of the total concentrate.

2. The concentrate of claim 1, wherein the total of the water and perfume oil(s) is greater than 99.5% by weight, based on the weight of the total concentrate.

3. The concentrate of claim 1, wherein the average droplet size of the emulsion d₅₀ is in the range of from greater than 0.1 μm to equal to or less than 5 μm.

4. The concentrate of claim 1, wherein the average droplet size of the emulsion d₅₀ is not larger than 400 nm.

5. The concentrate of claim 1, wherein the concentrate comprises at least 40% by weight of perfume oils.

6. The concentrate of claim 5, wherein the concentrate comprises at least 60% by weight of perfume oils.

7. The concentrate of claim 5, wherein the concentrate comprises equal to or less than 90% by weight of perfume oils.

8. The concentrate of claim 1, wherein the concentrate comprises less than 60% by weight of water

9. The concentrate of claim 1, wherein the concentrate comprises less than 40% by weight of water.

10. The concentrate of claim 1, wherein the concentrate comprises an emulsifier in a range of from at least 0.1% to equal to or less than 4% by weight based on the total concentrate.

11. The concentrate of claim 1, wherein the minimum amount of emulsifier is 0.2% by weight based on the total concentrate.

12. The concentrate of claim 1, wherein the emulsifier is selected from the group consisting of nonionic, zwitterionic, ampholytic, cationic and/or anionic emulsifiers.

13. The concentrate of claim 1 further comprising from at least 0.1% by weight to equal to or less than 4% by weight of a thickener based on the total concentrate.

14. The concentrate of claim 1, wherein the thickener is selected from the group consisting of
a) xanthan gum, guar derivatives, gum arabic, karaya gum, tragacanth, tara gum, gellan, carrageen, carob seed grain, agar agar, alginates, pectins and/or dextrans,
b) polyacrylates, polyacrylamides, polyvinylpyrrolidone, polvinyl alcohol, polyethylene glycols, hydrophobically modified polyethers, polyurethanes, styrene-maleic anhydride copolymers, their salts and/or derivatives,
c) hydroxyethylcellulose, carboxymethylcellulose, hydroxypropylmethylcellulose, hydroxypropylcellulose, ethylhydroxyethylcellulose, methylcellulose,
d) starch fractions and derivatives, in particular, amylose, amylopectin and dextrins,
e) bentonite.

15. The concentrate of claim 13, wherein the minimum amount of thickener is 0.2% by weight based on the total concentrate.

16. A method of producing a concentrate of claim 1 comprising the steps of:
a) adding a thickener to water while stirring the mixture in a first vessel,
b) adding an emulsifier to perfume oil with stirring in a second vessel,
c) adding the contents of the second vessel to the mixture of the first vessel with homogenization.

17. The method of claim 16, wherein step b) is carried out at temperatures below 60° C.

18. The method of claim 17, wherein the temperatures are in the range from 25 to 55° C.

19. The method of claim 16, wherein the mixture formed in step a) is heated to a temperature below of 50° C.

20. The method of claim 19, wherein the temperature Is in the range from 20 to 35° C.

21. A method of producing a concentrate of claim 1 comprising the steps of:
a) providing a vessel containing water;
b) adding a thickener and an emulsifier to the water with stirring;
c) adding a perfume oil to the thickener/emulsifier combination, wherein step c) is carried out with homogenization.

22. The method of claim 21, wherein the thickener is added at a temperature of below 60° C. followed by addition of the emulsifier during or after the heating of the thickener-water mixture to a temperature below 70° C., and then cooling the mixture to a temperature of below 50° C. before prior to step c).

23. The method of claim 21, wherein nitrogen and/or carbon dioxide is introduced into the liquid.

24. A product comprising a perfume oil concentrate of claim 1 and a solid in the form of a fine powder selected from the group consisting of zeolites, bentonites, silicates, phosphates, urea and/or derivatives thereof, sulfates, carbonates, citrates, citric acid, acetates and/or salts of the anionic surfactants.

25. A product comprising a perfume oil concentrate of claim 1 and a lipophilic thickener selected from the group consisting of fatty alcohols, fatty alcohol ethoxylates and/or derivatives thereof, fatty acids, fatty acid alkanolamide ethoxylates, paraffins and/or silicone oils.

26. The product of claim 25 comprising lipophilic thickeners in amounts of from 0.05 to 3% by weight based on the total perfume oil concentrate.

27. The product of claim 26, wherein the product exhibits a delayed scent effect.

28. A cosmetic product comprising a perfume oil concentrate of claim 1 and at least one cosmetically effective substance.

29. A textile treatment product comprising a perfume oil concentrate of claim 1 and a textile treatment substance.

30. A method of producing a product as claimed in claim 25, wherein a lipophilic thickener selected from the group consisting of fatty alcohols, fatty alcohol ethoxylates and/or derivatives thereof, fatty acids, fatty acid alkanolamide ethoxylates, paraffins and/or silicone oils is added to a perfume oil or perfume oil concentrate before or after the emulsification, wherein the amount of the thickener is from 0.05 to 3% by weight based on the perfume oil or the perfume oil concentrate.

31. The method of claim 30 further comprising the step of adding from 0.1 to 1 part by weight of emulsifier with homogenization, wherein the weight of the emulsifier is based on the perfume oil concentrate consisting of perfume oil emulsion and lipophilic thickener.
